(19) United States
(12) Patent Application Publication
(10) Pub. No.: US 2009/0105704 A1
(43) Pub. Date: Apr. 23, 2009

(54) COUNTERFORCE BRACE

(76) Inventor: Howard A. Gordon, JR., Kansas City, MO (US)

Correspondence Address: ERICKSON, KERNELL, DERUSSEAU & KLEYPAS, LLC, 800 W. 47TH STREET, SUITE 401, KANSAS CITY, MO, 64112, US

(21) Appl. No.: 12/276,745

(22) Filed: Nov. 24, 2008

Related U.S. Application Data
(63) Continuation-in-part of application No. 11/709,424, filed on Feb. 22, 2007, now abandoned.
Application No. 11/709,424 is a continuation-in-part of application No. 11/079,942, filed on Mar. 15, 2005.

(51) Int. Cl. A61F 5/00; A61B 19/00

(52) U.S. Cl. 606/27; 602/36; 128/898

(57) ABSTRACT

A stabilization brace presents a compressive, sock-like body for a compressive fit about the ankle joint and forefoot. A pair of force straps extends from the metatarsal heads and is wound in a preferred manner along opposed regions of the foot and ankle to offer countervailing forces therealong. First and second closure systems secure the body to the foot and offer circumferential support thereto. The brace resists excessive supination and pronation of the foot, ankle joint and underlying structures. Three embodiments of the brace are shown wherein one end of the force straps are attached to a zone adjacent the metatarsals as well as bifurcated ends attached to the metatarsal zone and a second zone spaced therefrom. A third embodiment presents bifurcated ends attached at one zone adjacent the metatarsals and another zone of the rear of the Achilles which presents an enhanced heel locking mechanism.

This application is a continuation-in-part of application Ser. No. 11/709,424, filed Feb. 27, 2007, which is a continuation-in-part of application Ser. No. 11/079,942, filed Mar. 15, 2005.

### BACKGROUND OF THE INVENTION

This invention relates to stabilization braces and more particularly to a support design which stabilizes the ankle and forefoot of the user at a desired neutral position.

The supination of a foot combines inward rotation at the ankle, adduction of the hind foot inversion of the forefoot and medial arch elevation. Basically, supination produces a medial (inward) movement at the talocalcaneal (subtalus) joint known as inversion as well as similar movement between the hind foot (talus) and mid foot (navicular) bones. A foot that supinates appears to naturally favor the lateral region or outside edge of the foot. A certain amount of supination is normal during the toe off stage of running, i.e., when the heel first lifts off the ground until the end of the step. During excessive supination, the lateral structures of the foot tighten which may rupture or avulse portions of the lateral collateral ligaments resulting in a “rolling over” of the ankle or ankle sprain.

The pronation of a foot produces opposite foot behavior of the subtalar joint including adduction of the forefoot, eversion of the hind foot and dorsiflexicon. Basically, pronation produces a lateral (outward) movement of the foot at the subtalar joint known as eversion as well as similar hind foot and mid foot movements. A foot that pronates appears to naturally favor the inside or medial edge of the foot. The foot naturally pronates to absorb the shock resulting from the heel hitting the ground. Excessive pronation increases the stress on the inside/medial portion of the foot which may lead to various injuries such as arch strain, heel pain, inner ankle pain and shin splints.

Accordingly, it is desirable to preclude excessive pronation and/or supination of the ankle joint and foot. Thus, various devices have been proposed to prevent ankle and foot injuries by attempting to maintain the foot in a “neutral position”. Although assumably effective in their operation, such devices have failed to simultaneously provide the effective stabilization of the ankle and forefoot and in a neutral position. This desired stabilization, which resists excessive pronation and/or supination, positions the foot for a proper impact during walking, running and jumping rather than on the edge of the foot while in an excessive pronated or supinated position.

In response thereto, stabilization braces are presented for simultaneously maintaining the ankle and the forefoot at a neutral position while permitting natural movement through a full range of motion. A first brace presents an elastic, sock-like body positioned in a compressive fit about the ankle and foot of the wearer. A pair of torque straps is anchored to the body adjacent the distal (head) ends of the first and fifth metatarsals. Each strap is then wound about the forefoot, heel and ankle and anchored above the ankle joint so as to present countervailing forces along the foot and ankle joint complex. These forces resist excessive movement of the foot and ankle from their normal, neutral position without precluding normal ambulatory movement thereof. The brace design anatomically fits either foot about the heel, ankle and forefoot aspects thereof. A first closure system which allows the sock-like body to be easily slipped onto the foot. A second closure system maintains the circumferential integrity of the brace about the ankle joint region and presents anchor zones for the free ends of the bilateral torque straps. This structure allows the user to variably adjust the tension on these torque straps and thus the countervailing forces presented thereby.

A second brace similar in construction presents bifurcated ends at the end of each torque strap. These ends are respectively anchored to the material body adjacent the distal ends of the metatarsals and the heel. Each strap is then wound about the foot as above with the dual anchor zones enhancing the distribution of opposing forces along the foot.

A third embodiment again presents bifurcated ends at the end of each torque strap. A first bifurcated end of each torque straps is respectively anchored to the material body adjacent the distal ends of the metatarsals with the second bifurcated end attached to the material body at the rear of the heel adjacent the Achilles tendon. The remaining portion of each strap, extending from the juncture of these bifurcated ends, is wound along the material body containing the foot as above described. The second bifurcated ends cooperate to present a single strap extending between medial and lateral locations on the forefoot and about the Achilles. The free ends of each strap are dually anchored along the medial and lateral sides of the ankle joint complex to Velcro® materials presented by the second bifurcated ends extending around the Achilles as well as the anchor zones on the second closure system as above described. This embodiment enhances the distribution of the forces along the foot and ankle joint complex. The plurality of anchor zones enhance the stability of the heel lock mechanism presented by the wound Achilles strap and torque straps.

It is therefor a general object of the invention to provide braces which simultaneously secure the ankle and the forefoot at a neutral position so as to resist excessive supination and pronation thereof.

Another object of the invention is to provide braces, as aforesaid, which present a pair of torque straps and method of wrapping the same so as to present countervailing forces between the metatarsal heads and the superior region of the ankle joint complex which resist excessive pronation or supination.

A particular of this invention is to provide braces, as aforesaid, which present a pair of torque straps and method of wrapping the straps either anchored at its ends to a zone adjacent the metatarsals, or spaced-apart zones adjacent the metatarsals and heel of the wearer, or adjacent the metatarsals and the Achilles of the wearer.

A further object of this invention is to provide braces, as aforesaid, having a sock-like body which slips over the foot and provides a compression fit thereto between the metatarsal heads and the superior region of the ankle joint complex.

Another object of this invention is to provide braces, as aforesaid, wherein a first closure system allows the brace to be used with variously sized feet while maintaining a compressive fit.

A still further object of this invention is to provide braces, as aforesaid, having a second closure mechanism for providing circumferential forces about the ankle joint complex while presenting anchor points for the torque straps.

Another particular object of this invention is to provide braces, as aforesaid, which presents a pattern to fit the ankle, heel and forefoot aspects of the foot of the wearer.

A more particular object of the invention is to provide a brace as aforesaid, which uses either heat seals along the seams of the brace pattern in lieu of stitching so as to enhance the fit of the brace and/or stitching.

A still more particular object of this invention is to provide braces, as aforesaid, which present reinforced stitching along the edges of the body, the edges of the closure mechanisms about a heel aperture in the sock-like body to preclude damage thereto during normal use.

Another particular object of this invention is to provide braces, as aforesaid, which present a plurality of anchor zones for the torque straps on the medial and lateral sides of the ankle joint complex to enhance the heel lock presented by the torque straps.

Another particular object of this invention is to provide torque straps, as aforesaid, which have elastic portions at least at the anchor zones adjacent the metatarsals to enhance a smooth wrapping of the torque wraps about the foot.

Other objects and advantages of this invention will become apparent from the following description taken in connection with the accompanying drawings, wherein is set forth by way of illustration and example, now preferred embodiments of this invention.

### BRIEF DESCRIPTION OF THE DRAWINGS

FIG. 1 is a top/dorsal view of the right foot of the wearer and showing in phantom lines the metatarsals and phalanges of the foot of the wearer;

FIG. 2 is a perspective view of the right foot of the wearer showing the sock-like body thereon, the unwrapped countervailing force straps (fragmentarily shown), the first closure system prior to full engagement and the second closure system disengaged from the ankle joint complex;

FIG. 3 is a view, as in FIG. 2, showing the FIG. 2 closure system in an engaged/locked position;

FIG. 4 is a view as in FIG. 3 showing the initial wrapping of the medial torque strap about the dorsal aspect of the foot, underneath the heel and along the medial side of the ankle joint complex prior to engagement of the free end of the medial strap to a superior position above the ankle joint complex;

FIG. 5 is an opposed perspective view of the foot in FIG. 4 showing the initial wrapping of the lateral torque strap across the dorsal aspect of the foot;

FIG. 6 is a view of the FIG. 5 foot showing the continuing engagement of the lateral strap across the dorsal aspect of the foot, underneath the heel extension of the lateral strap along the lateral side of the ankle joint complex prior to engagement of the free end of the lateral strap to a superior position above the ankle joint complex;

FIG. 7 is a bottom view of the foot showing the metatarsal anchor zone of the straps adjacent the free edge of the body and the extension of the straps wound under the heel;

FIG. 8 is a lateral view of the foot with brace secured thereon;

FIG. 9 is a view of the pattern of the brace showing the tongue element;

FIG. 10 is a view of the pattern of the brace showing the portion for encompassing the ankle joint;

FIG. 11 is a view of the pattern of the brace showing the portion for encompassing the heel and forefoot;

FIG. 12 is a view of one of the counter force straps;

FIG. 13 is a diagrammatic view showing pronation and supination of the right foot from the rear thereof;

FIG. 14 is a bottom view of the foot showing an alternative embodiment of the countervailing force straps presenting bifurcated ends anchored adjacent the metatarsals and heel of the wearer;

FIG. 15 is a perspective view of the wearer showing the sock-like body thereon and the initial wrapping of the medial strap about the dorsal aspect and underneath the foot of the wearer;

FIG. 16 is a lateral view of the foot with the alternative embodiment of the brace secured thereon;

FIG. 17 is a bottom view of the right foot showing an alternative embodiment of the force straps with bifurcated ends prior to wrapping;

FIG. 18 is a rear view of the right foot showing the straps of FIG. 17 prior to wrapping;

FIG. 19 is a lateral side of the foot showing the bifurcated ends of the lateral force strap in place at the initial wrapping thereof;

FIG. 20 is a front view of the device of FIG. 17 showing a portion of the medial and lateral straps extending therefrom with the second closure system disengaged;

FIG. 21 is a view, as in FIG. 20, showing a partial engagement of the second closure system;

FIG. 22 is a front view showing the initial wrapping of the lateral torque strap of FIG. 19 about the dorsal aspect of the foot;

FIG. 23 is a bottom view showing the wrapping of both torque straps along the underside of the foot;

FIG. 24 is a front view showing the wrapping of the torque straps about the dorsal and side aspects of the foot; and

FIG. 25 is a lateral side view of the foot, as in FIG. 22, showing the torque straps in place about the foot.

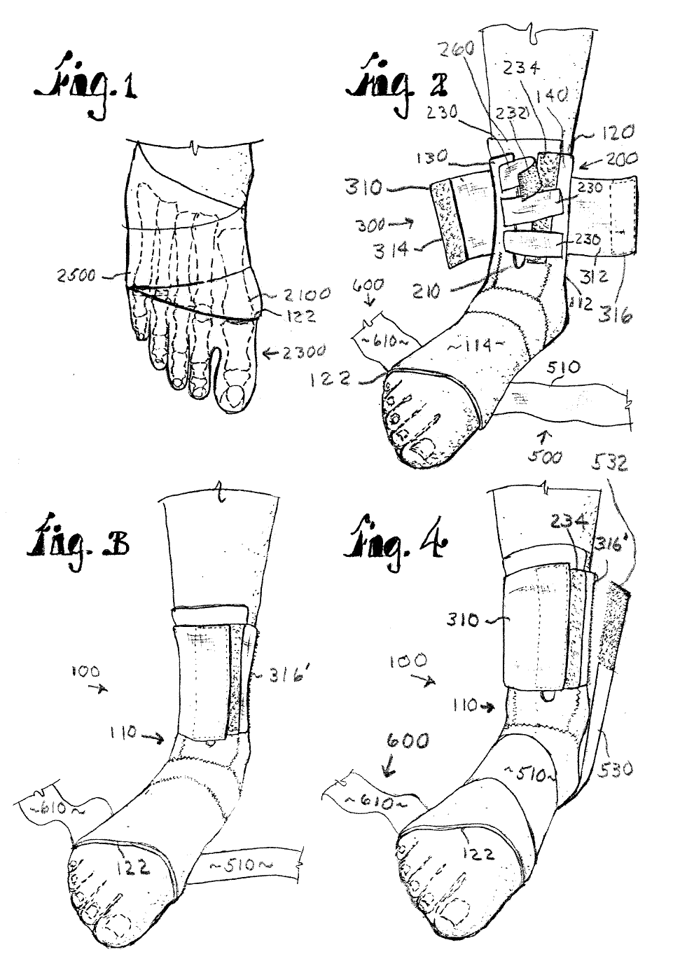

### DESCRIPTION OF THE PREFERRED EMBODIMENT

Turning more particularly to the drawings, the first brace 100 comprises a sock like body 110 made of an elastic material. The body 110 presents first 112 and second 114 integral sleeves made from joined blanks 112′, 114′. The body 110 provides a compression fit about the ankle joint complex and forefoot of the wearer. The forefoot is described as that region of the foot extending from the heel to approximate the distal/head ends of the first 2100 and fifth metatarsals 2500. The body 110 presents a first free edge 120 which circumscribes the foot at a superior position above the ankle joint complex. A second free edge 122 circumscribes the foot adjacent the distal ends/heads of the first 2100 and fifth 2500 metatarsals. It is preferred that this edge 122 not extend beyond such distal ends as it may present discomfort to the foot during flexion. As such, upon slipping the body 110 over the foot, the body 110 presents a compression fit between these edges 120, 122. Portions of the body 110 may be referred to in relation to the aspects of the foot it covers, e.g., dorsal, medial, lateral, etc.

The initial extension of the foot through the opening presented by the free edge 120 is enhanced by a closure system 200. This closure system is presented by a U-shaped notch 210 which extends from the free edge 120. As such, first and second flaps 130, 140 are presented which allow the user to increase the initial opening presented by the upper free edge 120. As such, a foot of various sizes may be initially extended through this free edge 120 for ultimate extension of the phalanges 2300 through the downstream free edge 122. Aperture 190 presents a seat for fitting of the heel therein and extension of a portion of the heel therethrough. The circumference of aperture 190 has been reinforced to resist fraying during use.

The closure system 200 presents a plurality of tabs 230 which are secured at one end along the longitudinal extent of the notch 210 on flap 130. These tabs have Velcro® fastener material 232 on the inside thereof. Complementary Velcro® material 234 is located along the extent of the notch 210 on the opposed flap 140. The engagement of each tab 230 fastener 232 to the complementary fastener 234 enables the user to adjust the compression of the sleeve 112 about the ankle joint region as the degree of overlap of these fasteners 232, 234 can be selectably adjusted. A tongue 260 is attached to the interior of the body portion 112 for extension along notch 210 so as to preclude the sleeve 112 and tabs 230 from directly contacting the user.

Further attached to the rear of body portion 112 adjacent edge 120 is a second closure system 300 which presents first 310 and second 312 straps made of an elastic and/or non-stretchable material. The straps 310, 312 present complementary Velcro® fasteners 314, 316 at the free ends thereof. Accordingly, these flaps are wrapped circumferentially around the ankle joint complex and attached one to the other as shown in FIG. 3. The edges of such closure system have been reinforced to prevent fraying during use. The degree of overlap of the complementary fasteners 314, 316 adjusts the compressive fit of straps 310, 312 about the ankle joint and thus maintains the circumferential integrity of the brace about the ankle joint complex. These straps 310, 312 further present Velcro® fasteners 316, 316′ along the lateral and medial sides of the ankle joint which mate with complementary Velcro® fasteners at the ends of the bilateral torque straps as to be further described.

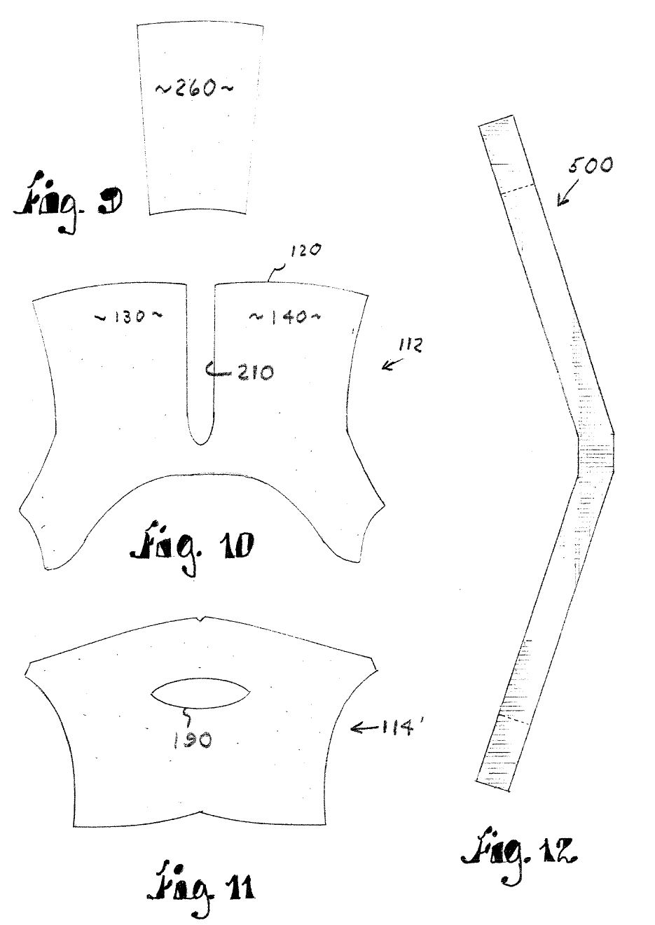

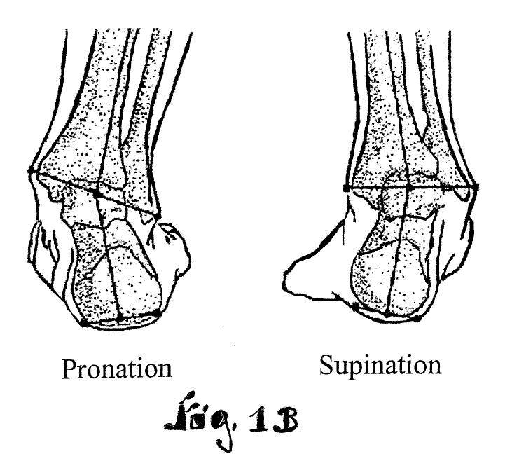

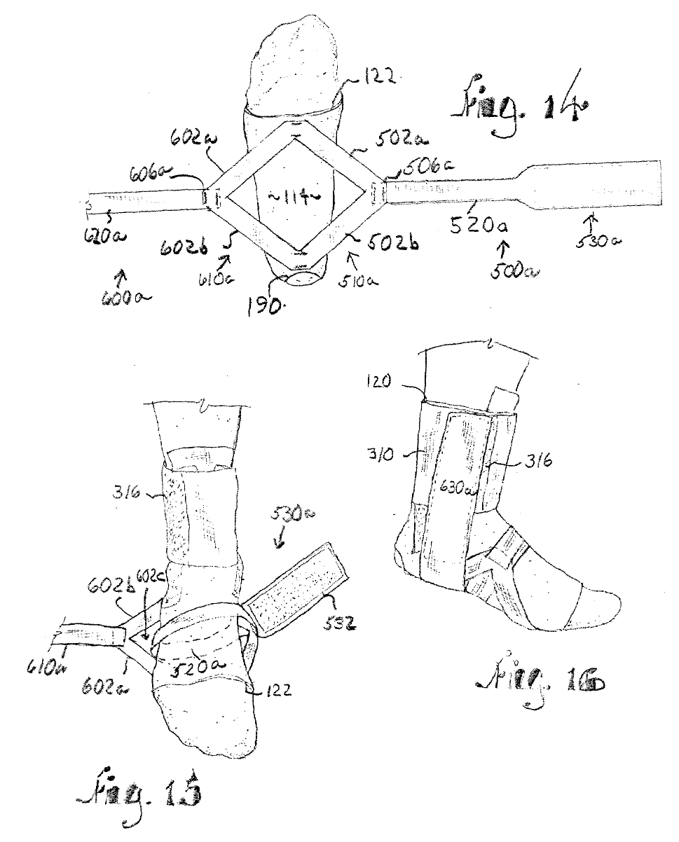

The brace 100 further includes medial 500 and lateral 600 countervailing force straps which are anchored at a location 550 underneath the sleeve 114 but may be otherwise anchored adjacent the free edge 122 and metatarsals (FIG. 7). These straps, 500, 600 are preferably made of a non-elastic material. However, I have found that the use of elastic material in a portion of the straps 500, 600, adjacent the anchor zones 550, enhances a smooth wrapping of the straps about the foot in a manner to be subsequently described. It is understood that the straps 500, 600 may be a single elongated strap anchored at 550 so as to present the medial 500 and lateral 600 straps or separate straps extending therefrom.

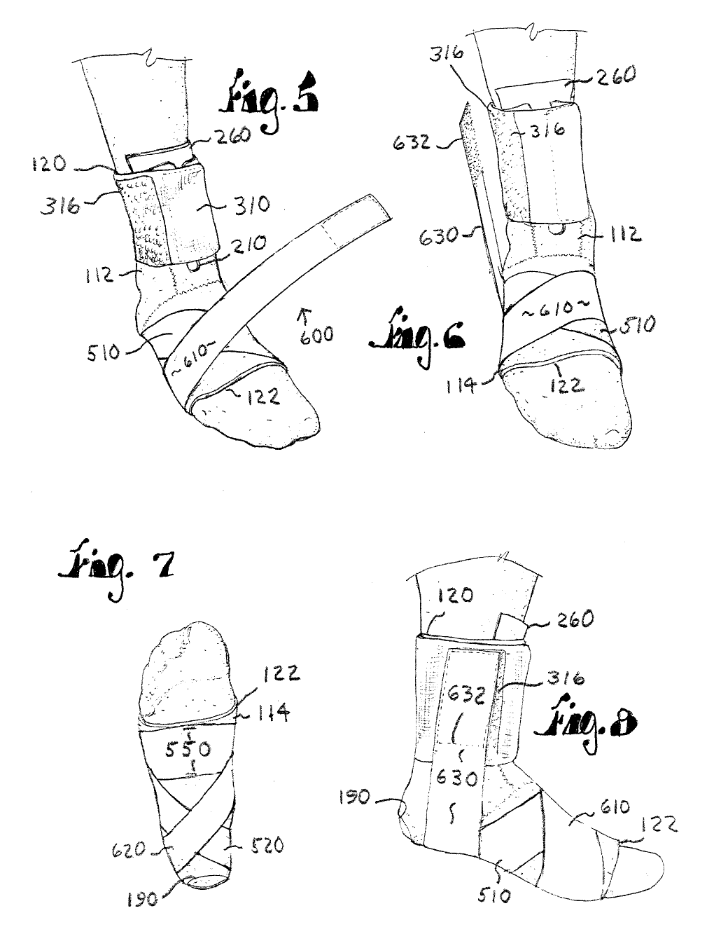

Accordingly, the right foot being shown, the medial strap 500 is first wrapped so as to provide a force along the forefoot, heel and ankle regions of the wearer. As such, a first extension 510 of strap 500 extends along body 110 from zone 550 and to the medial side of the foot, across the dorsal aspect of the forefoot and rearwardly towards the lateral side of the heel. A second extension of the strap 520 is wound underneath the body 110 covering the heel as best shown in FIG. 7. Subsequently, the remaining portion of the strap 530 is then brought continuously upwardly along the body 110 covering the medial side of the ankle joint region as best shown in FIG. 4. The end of the strap 500 presents Velcro® material 532 designed to mate with the Velcro® 316′ presented on flap 312 of the closure system 300 at a superior position above the ankle joint. As such, the wearer by pulling on strap 500 at extension 530 can variably adjust the mating of the strap 550 end to this Velcro® mating material 316′ and thus adjust the tension along the medial strap 500. In turn this tension is transmitted as a force along the medial and dorsal aspects at the foot, the lateral side of the heel, underneath the heel and along the medial side of the heel and ankle joint complex.

Subsequently, a first extension 610 of the lateral strap 600 is wound about the dorsal aspect of the foot from the lateral side thereof, across the dorsal aspect of the forefoot and rearwardly towards the medial side of the heel (FIGS. 5, 6). A second strap extension 620 is then wound underneath the heel (FIG. 7) with a third extension 630 brought upwardly along the lateral side of the heel and ankle joint (FIG. 8). The end of the strap extension 630 likewise presents a Velcro® fastener 632 which engages complementary material 316 presented on strap 310 of the closure system 300. Again, the variable anchoring of the strap extension 630 to this Velcro® material 316 enables the user to adjust the tension along this strap 600. In turn this tension is transmitted as a force, opposite that force presented by strap 500, along the lateral and dorsal aspects at the foot, the medial side of the heel, underneath the heel and along the lateral side of the ankle joint complex.

Accordingly, the body 110 along with the closure systems 200, 300 provides compressive forces about the ankle and foot of the user, inclusive of a heel lock mechanism therein. The method of winding the medial 500 and lateral 600 straps presents countervailing forces beginning at the metatarsal heads, along the forefoot and upward along the ankle joint complex. As previously discussed, pronation and supination of the foot respectively present opposed movements at the subtalar joint, hind foot and mid foot such that the foot favors the lateral region foot (supination) or the medial region (pronation) of the foot. Straps 500, 600 thus present countervailing forces in these regions so as to resist movement of the ankle and foot, inclusive of its underlying structures, from excessive pronation or supination. Thus, as the foot is held in a neutral position, the probability of the user landing on an excessively supinated or pronated foot during walking, jumping and/or running is reduced which reduces the probability of injury. The wrapping of these counter force straps 500, 600 with or without the compressive fit offered by the body 110 and the compression mechanisms 200, 300 enhances the prophylactic advantages of the brace 100.

Alternatively, a second embodiment of brace 100a is shown in FIGS. 14-16. Brace 100a presents alternative medial 500a and lateral 600a force straps. The end of the straps 500a, 600a present bifurcated ends 502a, 502b, 602a, 602b. The strap ends 502a, 602a are shown to be anchored underneath the sleeve 114 adjacent the free edge 122 but may be otherwise anchored on the sleeve adjacent the metatarsals. The ends 502b, 602b are anchored underneath the sleeve 114, adjacent the edge presented by heel aperture 190 and thus underneath the heel but may be otherwise on the sleeve adjacent the heel. These bifurcated ends are included in a first extension 510a, 610a of each strap and function as the first extensions 510, 610 explained hereinabove. Subsequent extensions 520a, 620a, 530a, 630a with Velcro® material thereon function as above described in embodiment 100.

Embodiment 100a is utilized in a manner as above described in embodiment 100. As such, the foot is initially extended through the opening 120 presented by the free edge of the body member 110 for ultimate extension of the metatarsal flanges 2300 through this free edge and the downstream free edge 122 with extension of the heel through aperture 190. First and second closure systems 200 and 300 function as above described.

The medial strap 500a is first wrapped so as to provide a force along the forefoot, heel and ankle regions of the wearer. As such, the free end of the strap 500a is first extended through the opening 602c presented by the bifurcated strap ends 602a, 602b of lateral strap 600a. The first extension 510a of strap 500a, comprising, in the main, ends 502a, 502b, extend from the two spaced anchor points on sleeve 114 to the medial side of the foot across the dorsal aspect of the forefoot and rearwardly towards the lateral side of the foot.

A second extension 520a of the strap comprising, in the main, the reduced lateral portion of strap 520a is wound underneath the foot and shown in phantom in FIG. 15. The remaining portion of the strap 530a is brought along the medial side of the ankle joint such that the end of the strap can mate the Velcro® material 532 thereon with the Velcro® material 316′ presented on the flap 312 of the closure system. As above, the wearer by pulling on the strap 500a at extension 530a can adjust the mating of the strap 530a to the Velcro® 316′ and thus the tension along the medial strap 500a. This tension is transmitted along the medial dorsal aspects of the foot, the lateral side of the heel, the medial side of the heel and ankle joint and along the underneath of the heel as presented by the dual anchor points of the bifurcated strap ends 502a, 502b.

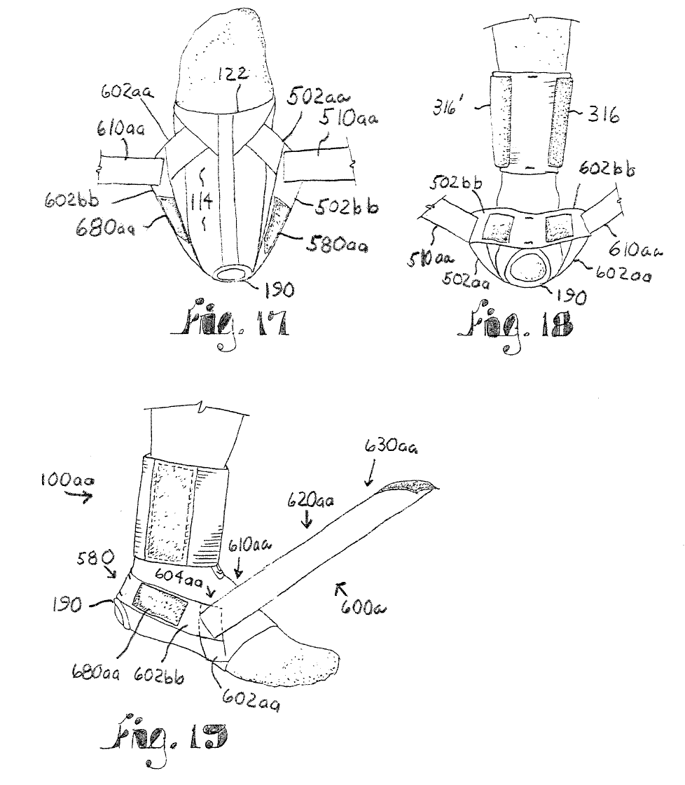

Strap 600a works on foot in an opposed manner to present countervailing forces on the foot. Thus, the first extension 610a of the lateral strap 600a including, in the main, the bifurcated straps 602a, 602b, is wound about the dorsal aspect of the foot from the lateral side thereof, across the dorsal aspect of the foot and rearwardly toward the medial side of the heel. A second strap extension 620a, i.e., primarily the reduced lateral portion, is then wound underneath the heel with a third extension 630a brought upwardly along the lateral side of a heel and ankle joint complex (FIG. 16). The end of the strap 630a likewise presents a Velcro® fastener which engages complementary material 316 presented on strap 310 of closure system 300. The variable Velcro® anchoring of this strap extension 630a allows users to adjust the tension of the strap 600a which is transmitted as a force opposite that presented by strap 500a in a manner as described in embodiment 100. Due to the bifurcated strap ends 602a, 602b and dual anchor zones, forces are presented along the underneath of the foot. Accordingly, I have found that such a brace 100a presents an enhanced leverage along all aspects of the foot.

It is also noted that the bifurcated ends of one of the straps, e.g., 602a, 602b, present a guide 602c for the initial extension of the free end of the opposite strap, e.g., 500a, so as to direct the initial winding of the first strap, e.g., 500a, in a proper direction. Thus, the proper application of the countervailing force straps 500a, 600a about the foot of the wearer is assured.

A third embodiment 100aa is as shown in FIGS. 17-25 and utilizes the principles, inclusive of the wrapping method, disclosed in the first two embodiments 100, 100a. Again, this third embodiment 100aa presents alternative medial 500aa and lateral 600aa torque straps. The end of these straps again presents first and second bifurcated branches/ends 502aa, 502bb, 602aa, 602bb. The strap branches 502aa, 602aa are anchored to the medial or lateral sides of sleeve 114 at a position adjacent the free edge 122 and the metatarsals. In this case the respective ends 502aa, 602aa are attached to the portion of the material body 110 adjacent the metatarsals and covering the medial and lateral sides of the forefoot. The branches 502aa, 602aa are preferably made of an elastic material to assure a smooth wrapping of the straps 500aa, 600aa about the material body and foot therein.

The respective strap ends also include bifurcated ends or branches 502bb, 602bb extending from the respective first branch 502aa, 602aa. Thus, junctures 504aa, 604aa are presented. These second branches 502bb, 602bb rearwardly extend from their respective junctures 504aa, 604aa and toward the rear of the material body. They are preferably affixed to the rear of the material body covering the rear of the heel located above the aperture 190 at 580. Aperture 190 provides a seat for the heel when the body 110 is slipped over the foot as previously described.

As shown, these second branches 502bb, 602bb can be in the form of a continuous strap extending between the respective junctures 504aa, 604aa and about the rear of the heel. As such, this strap bears against the Achilles of the wearer which enhances the resulting heel lock mechanism when the straps 500aa, 600aa are properly wound in place. As shown, each of these branches 502bb, 602bb have Velcro® mating material thereon 580aa, 680aa for a purpose to be subsequently described.

Embodiment 100aa is utilized in a manner as above described in embodiment 100, 100a. (In this embodiment 100aa an initial closure system 200′ is used which utilizes a Velcro® closure system 233 instead of tabs 230 as shown in FIG. 2. Velcro® 231 is presented for mating with Velcro® on flap 312.) As such the foot is initially extended through the opening 120 presented by the free edge of the body member 110 for ultimate extension of the metatarsal flanges 2300 through this free edge 120 and the downstream free edge 122 with the heel being fitted in the seat provided by aperture 190. The closure systems 200, 300 function as above described. Reinforcing stays (not shown) have been inserted in the flaps 310, 312 of system 300.

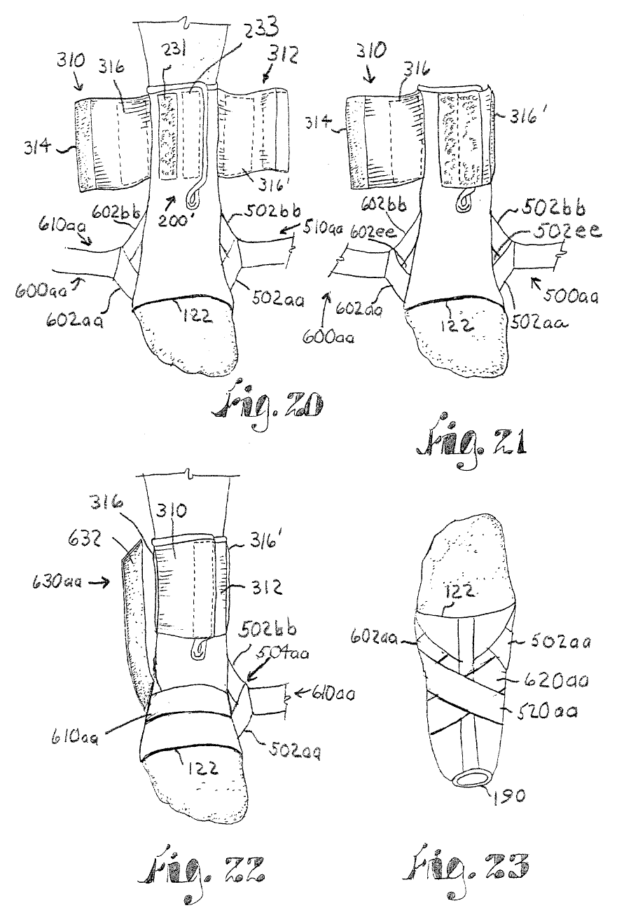

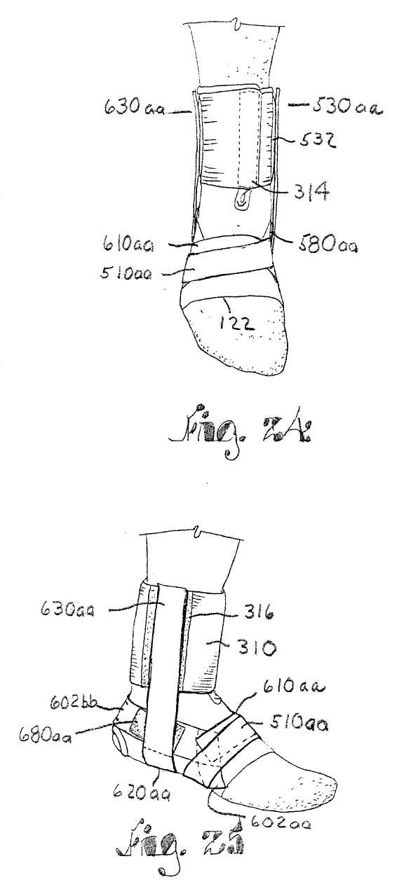

Although either strap 500aa, 600aa may be first wrapped the lateral strap 600 is shown as first wrapped (FIG. 19). As such the free end of the strap 600aa is first extended through the opening 502ee presented by the bifurcated branches 502aa, 502bb of the medial strap 500aa. The first longitudinal portion 610aa extends from the juncture 604aa on the lateral side of the foot, across the dorsal aspect of the forefoot and rearwardly towards the medial side of the foot (FIG. 22). Upon such positioning the bifurcated ends 602aa, 602ee are drawn against the body 110 covering the lateral side of the foot (FIG. 25).

A second portion 620aa of the strap is then wound along the body of material covering the underside of the foot from the medial to the lateral side thereof (FIG. 23). The remaining or third portion of the strap 630aa is then vertically extended along the lateral side of the ankle joint such that the end 630aa of strap 600aa presents the Velcro® material 632 thereon for complementary engagement with the Velcro® material 316 presented on the adjacent flap 312 of the closure system (FIG. 22). This mating occurs at a superior position above the ankle joint on the lateral side thereof. As above, the wearer by pulling on the strap extension 630aa can adjust the degree of mating of the strap portion 630aa to the Velcro® 316 which adjusts the tension along the lateral strap 600aa. Furthermore, the Velcro® material 632 on the strap end 630aa mates with Velcro® material 680aa on the second bifurcated branch 602bb of the lateral strap 600aa which further maintains the tension in strap 600aa. It also assists in maintaining the heel lock partially presented by strap 600aa. Accordingly, the tension is transmitted along the strap 600aa and thus along the foot and ankle joint complex. The arrangement of the straps initiates the beginning of a heel locking mechanism.

Strap 500aa is wrapped about the foot in an opposed manner. Thus, the first longitudinal extension 510aa of the lateral strap 500aa is wound about the dorsal aspect of the foot and rearwardly toward the lateral side of the heel. The second strap extension 520aa is then wound underneath the heel from the lateral to medial side thereof (FIG. 23) with a third extension 530aa brought continuously upwardly along the medial side of the heel and ankle joint complex (FIG. 24). The end of the strap portion 530aa likewise presents Velcro® fasteners 532 which engage complementary Velcro® material 316′ presented on flap 310 of closure system 300 as well as complementary Velcro® material 580aa presented on the second bifurcated branch 502bb. Again, the variable Velcro® anchoring of this strap extension 530aa allows users to adjust the tension along the strap 500aa which is transmitted as a force opposite that presented by strap 600aa in a manner as described in the above embodiments. Due to the opposed bifurcated strap ends, dual anchor zones and opposing longitudinal extension of each strap enhanced forces are presented along the opposed sides of the foot.

At this time a continuous Achilles strap, as presented by the aligned bifurcated branches 502bb, 602bb, is secured in its bearing relationship against the medial and lateral sides of the foot with Achilles therebetween which resists undesirable Achilles movement. Accordingly, the heel locking mechanism presents a double heel lock which resists undesirable movement of the heel and Achilles area. Accordingly, brace 100aa presents a further enhanced leverage along all aspects of the foot within the material body.

It is noted that embodiment 100aa is similar in construction to embodiments 100, 100a. Thus, identical numbers refer to identical elements as utilized between the two braces 100, 100a.

It is to be understood that while certain forms of this invention have been illustrated and described, it is not limited thereto, except in so far as such limitations are included in the following claims and allowable equivalents thereof.

Having thus described the invention, what is claimed as new and desired to be secured by Letters Patent is:

1. A stabilizing brace comprising:
a body of elastic material having a sock-like configuration configured to encompass an ankle joint and a portion of a forefoot of a wearer positioned therein, said body presenting:
a first free edge presenting a first aperture for initial extension of a wearer's foot therethrough;
a second free edge presenting a second aperture adapted for a subsequent extension of the wearer's foot therethrough, said material body extending between said first and second free edges, said first free edge encompassing the positioned foot of the wearer at a superior location above the ankle joint with said second free edge encompassing the positioned foot of the wearer at the distal ends of the metatarsals of the foot upon said extension of the wearer's foot therethrough, said body of material extending between said free edges;
a heel seat presenting an area for reception of the wearer's heel upon said positioning of the foot within said body of material;
a first elongated strap having at least a first end anchored adjacent said second free edge of said body, said first strap having a length comprising:
a first portion extending from said anchored end and across a portion of said material body covering a dorsal aspect of the foot to a lateral position on said material adjacent said heel seat;
a second integral portion extending from said first portion at said lateral position, along a portion of said body of material covering the heel and to a medial side of the body of material covering the heel;
a third integral portion extending from said second portion at the medial side of the heel area and generally continuous along a medial side of the body of material covering the ankle joint, said third portion presenting a free end;
first means for attaching said free end of said third portion of said first strap to said material body on the medial side thereof at a position adjacent said first free edge, whereby to tension said first strap;
a second elongated strap having at least a first end anchored adjacent said second free edge of said body, said second strap having a length comprising:
a first portion extending from said anchored end of said second strap and across a portion of said material body covering the dorsal aspect of the body to a medial position on said material body adjacent said heel seat;
a second integral position extending from said first portion of said second strap at said medial position, along said body of material covering the heel and to a lateral side of the body of material covering the heel;
a third integral portion extending from second portion of said second strap at the lateral side of the heel and generally continuous along a lateral side of the body of material covering the ankle joint, said third position of said second strap presenting a free end;
second means for attaching said free end of said third portion of said second strap to said material body on the lateral side thereof at a position adjacent said first free edge, whereby to tension said second strap, said first and second straps presenting countervailing forces along the foot and ankle joint positioned within said material body to resist excessive supination and pronation thereof.

2. The brace as in claim 1 wherein said first attaching means comprises:
a first fastener element on said third portion of said first strap;
a second complementary fastener element on said body at a superior position above the ankle joint, said engagement of said first and second fastener elements attaching said third portion of said first strap to said body.

3. The brace as in claim 2 wherein said second attaching means comprises:
a third fastener element on said third portion of said second strap;
a fourth complementary fastener element on said body at a superior position above the ankle joint, said engagement of said third and fourth fastener elements attaching said third portion of said second strap to said body.

4. The brace as in claim 1 wherein said first and second straps are anchored to said body adjacent a sole of the wearer.

5. The brace as in claim 1 further comprising:
a notch in said body portion extending from said first free edge, said notch presenting first and second flaps, each flap including a portion of said first free edge, a manipulation of said flaps adjusting a size of said first aperture presented by said first free edge.

6. The brace as in claim 5 further comprising:
means for maintaining said flaps in place about the ankle joint of the wearer.

7. The brace as in claim 5 further comprising:
a tongue spanning said notch.

8. The brace as in claim 1 further comprising:
a third strap having an end attached to said body at a position adjacent said first free edge;
a fourth strap having and end attached to said body at a position adjacent said first free edge, said third and fourth straps adapted to encircle the ankle joint region of the wearer; and
means for engaging said third and fourth straps.

9. The brace as in claim 8 wherein said engaging means comprises:
a fastener at a free end of said third strap;
a complementary fastener element at a free end of said fourth strap, said fastener elements on said third and fourth straps engageable one to the other to regulate a degree of said support offered by said third and fourth straps about the ankle joint.

10. The brace as in claim 5 further comprising:
a third strap attached to said body at a position adjacent said first free edge;
a fourth strap attached to said body at a position adjacent said first free edge, an engagement of said third and fourth straps adapted to cover said notch and encircle the ankle joint to offer support thereto.

11. The brace as claimed in claim 10 further comprising:
a fastener at a free end of said third strap;
a complementary fastener element at a free end of said fourth strap, said fastener elements on said third and fourth straps variably engageable one to the other to provide said engagement of said third and fourth straps about the ankle joint.

12. The brace as claimed in claim 1 wherein said at least first end of each of said straps comprises first and second bifurcated branches, said first branch of each strap presenting said at least first end of each strap anchored adjacent said second free edge of said body, said second branch of each strap anchored at a position on said material body displaced from said second free edge, said first and second branch of each strap presenting a juncture.

13. The brace as claimed in claim 12 wherein said second branches of each strap are anchored to a portion of said material body covering the wearer's heel.

14. The brace as claimed in claim 12 wherein said second branches area anchored to a portion of said material body covering the Achilles of the wearer.

15. The brace as claimed in claim 12 further comprising means on said respective second branches for mating with an adjacent portion of said third portion of said respective strap.

16. A method of stabilizing an ankle joint and foot of a wearer comprising the steps of:
positioning a body of elastic material having a sock-like configuration about an ankle joint and a foot of a wearer, said positioning presenting:
a first free edge in said body of material encompassing the ankle of the wearer at a superior location above the ankle joint;
a second free edge in said body of material encompassing the foot of a wearer at the distal ends of the metatarsals of the foot, said positioned body extending between said free edges and about the ankle joint, heel and foot of the wearer;
providing a first strap having at least a first end anchored adjacent said second free edge of said material body;
extending in contact with said body of material a first portion of said first strap from a portion of said material body covering a medial sided of the foot and across said material body covering the dorsal aspect of the foot to a lateral position on the material body adjacent a wearer's heel;
extending in contact with said body of material a second following portion of said first strap from said lateral position on said material body across said material body covering the underside of the heel and to a position on the material body covering a medial portion of the heel;
extending in contact with said body of material a third portion of said strap following said second portion from said medial position at the heel and continuously along the material body covering a medial side of the ankle joint to a superior location thereabove;
attaching a free end of said third portion of said first strap to said material body at the superior position above the ankle joint to tension said first strap;
providing a second elongated strap with at least a first end of said second strap anchored adjacent said second free edge of said material body;
extending in contact with said body of material a first portion of said second strap from a generally medial position on the material body adjacent the wearer's heel;
extending in contact with said body of material a second following portion of said second strap from said medial position of said material body across said material body covering the underside of the heel and to a position of the material body covering a lateral portion of the heel;
extending in contact with said body of material a third portion of said second strap following said second portion of said second strap from said lateral position and continuously along the material body covering a lateral side of the ankle joint to a superior location thereabove;
attaching a free end of said third portion of said second strap to said material body at a superior position above the ankle joint positioned to tension said second strap, said first and second tensioned straps presenting countervailing forces along the foot and ankle joint positioned within said material body to resist excessive supination and pronation thereof.

17. The method as claimed in claim 16 wherein each strap present first and second bifurcated ends, said first bifurcated end of each strap presenting said at least first strap end, and further comprising the step of affixing each second bifurcated end to said material body at a position displaced from said first bifurcated end.

18. The method as claimed in claim 17 wherein the step of affixing each second bifurcated end comprises affixing each bifurcated end to said material body at a portion thereon covering the Achilles.

19. The method as claimed in claim 18 further comprising the step of integrating said second bifurcated ends to present a continuous strap extending along said material body between each of said first bifurcated ends of each respective strap and about the rear of the Achilles.

20. The method as claimed in claim 18 further comprising the steps of:
attaching said third portion of each strap to adjacent each second bifurcated end, whereby to maintain said respective third portion of each strap in place along said body of material covering said lateral and medial sides of the ankle joint.

21. A method of stabilizing an ankle joint and foot of a wearer comprising the steps of:
providing a first elongated strap having at least a first end;
maintaining said at least first end to a position adjacent the metatarsals of the foot of the wearer;
extending along said foot a first portion of said first strap from a generally medial side of the foot and across a dorsal aspect of the foot to a lateral position on the foot adjacent a wearer's heel;
extending a second following portion of said first strap from said lateral position on said foot, across the underside of the heel and to a medial position on the foot adjacent the heel;
extending in a third position of said strap following said second portion from said medial position at the heel and continuously along a medial side of the ankle joint to a superior location thereabove;
maintaining a free end of said third portion of said first strap at the superior position above the ankle joint to tension said first strap;
providing a second elongated strap having at least a first end;
maintaining said at least first end of said second strap to a position adjacent the metatarsals of the foot of the wearer;
extending along said foot a first portion of said strap from a generally lateral side of the foot and across the dorsal aspect of the foot to a medial position adjacent the wearer's heel;
extending a second following portion of said second strap from said medial position on the foot, across the underside of the foot and to a lateral position of the foot adjacent the heel;
extending a third portion of said second strap following said second portion of said second strap from said lateral position and continuously along a lateral side of the ankle joint to a superior location thereabove;
maintaining a free end of said third portion of said second strap at said superior position above the ankle joint positioned to tension said second strap, said first and second tensioned straps presenting countervailing forces along the foot and ankle joint to resist excessive supination and promotion thereof.

22. The method as claimed in claim 21 further comprising the step of first positioning a body of elastic material having a sock-like configuration about the ankle joint and foot of the wearer, said maintaining and extending straps winding said respective straps about said body of material.
